(19) United States
(12) Patent Application Publication
(10) Pub. No.: US 2009/0170902 A1
(43) Pub. Date: Jul. 2, 2009

(54) COMPOUNDS WHICH POTENTIATE GLUTAMATE RECEPTOR AND USES THEREOF IN MEDICINE

(76) Inventors: Daniele Andreotti, Verona (IT); Simon Edward Ward, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/278,604

(22) PCT Filed: Feb. 6, 2007
(86) PCT No.: PCT/EP2007/051136

§ 371 Date: Dec. 3, 2008

(30) Foreign Application Priority Data
Feb. 8, 2006 (GB) 0602560.5

(51) Int. Cl. A61K 31/443; C07D 307/02; C07D 405/00; A61K 31/34

(52) U.S. Cl. 514/336; 549/480; 546/284.7; 514/472

(57) ABSTRACT

Compounds of formula (I), salts and solvates thereof are provided:

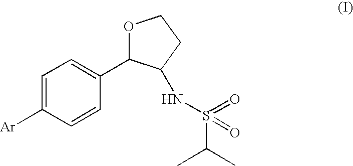

wherein Ar is selected from phenyl, pyridyl, furanyl and thienyl, each optionally substituted with one or more groups Y; and each Y group is independently selected from the group consisting of: halo, C₁₋₄alkyl, haloC₁₋₄alkyl, C₁₋₄alkoxy, cyano, C(O)C₁₋₄alkyl, NHSO₂C₁₋₄alkyl, NMeSO₂C₁₋₄alkyl, NHCOC₁₋₄alkyl, NMeCOC₁₋₄alkyl, SOC₁₋₄alkyl, SO₂C₁₋₄alkyl, and CO₂C₁₋₄alkyl, or two Y groups together form a cyclic group —O(CH₂)O—. Processes for preparation, and uses thereof in the treatment of a disease or condition mediated by a reduction or imbalance in glutamate receptor function, such as schizophrenia or cognition impairment, are also disclosed.

This invention relates to novel compounds which potentiate the glutamate receptor. The invention also relates to the use of the compounds in treating diseases and conditions mediated by potentiation of the glutamate receptor, compositions containing the derivatives and processes for their preparation.

Glutamate receptors, which mediate the majority of fast excitatory neurotransmission in the mammalian central nervous system (CNS), are activated by the excitatory amino acid, L-glutamate (for review see Watkins J C, Krogsgaard-Larsen P, Honore T (1990) Trends Pharmacol Sci 11: 25-33).

Glutamate receptors can be divided into two distinct families. The G-protein or second messenger-linked “metabotropic” glutamate receptor family which can be subdivided into three groups (Group I, mGlu1 and mGlu5; Group II, mGlu2 and mGlu3; Group III, mGlu4, mGlu6, mGlu7, mGlu8) based on sequence homology and intracellular transduction mechanisms (for review see Conn P J and Pinn J P (1997) Ann Rev Pharmacol Toxicol 37: 205-237). The “ionotropic” glutamate receptor family, which directly couple to ligand-gated cation channels, can be subdivided into at least three subtypes based on depolarizing activation by selective agonists, N-methyl-D-aspartate (NMDA), α-amino-3-hydroxy-5-methylisoxazole-4-propionic acid (AMPA) and kainic acid (KA) (for review see Dingledine R, Borges K, Bowie, Traynelis S (1999) 51: 7-61).

Native AMPA receptors (AMPAR) exist as heterotetramers consisting of combinations of four different protein subunits (GluR1-4) (for review see Bettler B and Muller C (1995) 34: 123-139.). Receptor subunit diversity is increased further as each subunit can undergo alternative splicing of a 38 amino acid sequence in the extracellular region just before the fourth membrane spanning domain M4. Such editing results in so-called ‘flip’ and ‘flop’ receptor isoforms which differ in kinetic and pharmacological properties (Sommer B, Keinanen K, Verdoon T A, Wisden W, Burnashev N, Herb A, Kohler M, Takagi T, Sakmann B, Seeburg P H (1990) Science 249: 1580-1585).

Additionally, post-transcriptional editing of GluR2 mRNA changes a neutral glutamine to a positively charged arginine within M2. In normal humans >99% GluR2 is edited in this way. AMPAR containing such edited GluR2 subunit exhibit low calcium permeability (Burnachev N, Monyer H, Seeburg P H, Sakmann B (1992) Neuron 8: 189-198). There is a suggestion, however, that the number of AMPAR with high calcium permeability is elevated in certain disease-associated conditions (Weiss J H, and Sensi S L (2000) Trends in Neurosci 23: 365-371).

AMPAR depolarization removes voltage dependent Mg²⁺ block of NMDA receptors which in turn leads to NMDA receptor activation, an integral stage in the induction of Long-Term Potentiation (“LTP”) (Bliss T V P, Collingridge G L (1993) Nature 361: 31-9). LTP is a physiological measure of increased synaptic strength following a repetitive stimulus or activity, such as occurs during learning.

It has been reported that direct activation of glutamate receptors by agonists, in conditions where glutamate receptor function is reduced, increases the risk of excitotoxicity and additional neuronal damage. AMPAR positive allosteric modulators do not activate the receptor directly. However, when the ligand (L-glutamate or AMPA) is present AMPAR modulators increase receptor activity. Thus, AMPA receptor modulators enhance synaptic function when glutamate is released and is able to bind at post-synaptic receptor sites.

Compounds which act as AMPAR positive allosteric modulators have been shown to increase ligand affinity for the receptor (Arai A, Guidotti A, Costa E, Lynch G (1996) Neuroreport. 7: 2211-5.); reduce receptor desensitization and reduce receptor deactivation (Arai A C, Kessler M, Rogers G, Lynch G (2000) 58: 802-813) and facilitate the induction of LTP both in vitro (Arai A, Guidotti A, Costa E, Lynch G (1996) 7: 2211-5.) and in vivo (Staubli U, Perez Y, Xu F, Rogers G, Ingvar M, Stone-Elander S, Lynch G (1994) Proc Natl Acad Sci 91: 11158-11162). Such compounds also enhance the learning and performance of various cognitive tasks in rodent (Zivkovic I, Thompson D M, Bertolino M, Uzunov D, DiBella M, Costa E, Guidotti A (1995) JPET 272: 300-309, Lebrun C, Pilliere E, Lestage P (2000) Eu J Pharmacol 401: 205-212), sub-human primate (Thompson D M, Guidotti A, DiBella M, Costa E (1995) Proc Natl Acad Sci 92: 7667-7671) and man (Ingvar M, Ambros-Ingerson J, Davis M, Granger R, Kessler M, Rogers G A, Schehr R S, Lynch G (1997) Exp Neurol 146: 553-559).

Compounds which act as AMPAR positive allosteric modulators are known, for example in international patent application WO2006/015828. We have discovered a class of novel compounds that also potentiate the AMPA receptor.

According to a first aspect, the invention provides a compound of formula (I), or a salt, or solvate thereof:

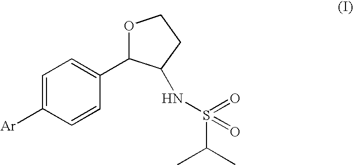

wherein:
Ar is selected from phenyl, pyridyl, furanyl and thienyl, each optionally substituted with one or more groups Y;
each Y group is independently selected from the group consisting of: halo, C₁₋₄alkyl, haloC₁₋₄alkyl, C₁₋₄alkoxy, cyano, C(O)C₁₋₄alkyl, NHSO₂C₁₋₄alkyl, NMeSO₂C₁₋₄alkyl, NHCOC₁₋₄alkyl, NMeCOC₁₋₄alkyl, SOC₁₋₄alkyl, SO₂C₁₋₄alkyl, and CO₂C₁₋₄alkyl, or two Y groups together form a cyclic group —O(CH₂)O—.

As used herein, the terms “halogen” and its abbreviation “halo” refer to fluorine, chlorine, bromine, or iodine.

C₁₋₄alkyl may be a straight chain or branched alkyl group. For example, a C₁₋₄alkyl group may be selected from methyl, ethyl, n-propyl, i-propyl, n-butyl, i-butyl or t-butyl. For example, C₁₋₄alkyl is methyl, or “Me”.

As used herein, the term “C₁₋₄alkoxy” refers to the group —O—C₁₋₄alkyl wherein C₁-C₄alkyl is as defined above.

As used herein, the term “haloC₁₋₄alkyl” refers to a C₁₋₄alkyl group as defined above which is substituted with any number of fluorine, chlorine, bromine, or iodine atoms, including with mixtures of those atoms. A haloC₁₋₄alkyl group may, for example contain 1, 2 or 3 halogen atoms. For example, a haloC₁₋₄alkyl group may have all hydrogen atoms replaced with halogen atoms. Examples of haloC₁₋₄alkyl groups include fluoromethyl, difluoromethyl and trifluoromethyl.

In an embodiment the salt or solvate of the compound of formula (I) is a pharmaceutically acceptable salt or solvate. In one embodiment, the invention provides a compound of formula (I), a pharmaceutically acceptable salt or solvate thereof.

In an embodiment, Ar is selected from phenyl or pyridyl substituted with one or more groups Y; or thienyl optionally substituted with one or more groups Y.

In an embodiment, Ar is selected from phenyl and pyridyl. In a further embodiment, Ar is pyridyl.

In an embodiment when Ar is a pyridyl group, the pyridyl group is position with the ring nitrogen meta or para to the point of attachment to the remainder of the molecule. In a further embodiment, the pyridyl nitrogen is meta to the point of attachment to the remainder of the molecule.

In one embodiment, zero, one, two or three Y groups are present.

In an embodiment, each Y is selected from is independently selected from the group consisting of: halo, methyl, cyano and C(O)CH₃. In a further embodiment, each Y is independently selected from the group consisting of: fluoro, methyl, cyano and C(O)CH₃.

In an embodiment, the molecule has one Y group. In one embodiment, the one Y group is selected from fluorine and methyl. In one embodiment the one Y group is fluorine.

Each substituent Y may be positioned ortho, meta or para to the point of attachment of the group Ar to the remainder of the molecule. In one embodiment a Y group is positioned para or meta with respect to the point of attachment of the Ar group to the main structure of the molecule. In a further embodiment, a Y group is positioned meta with respect to the point of attachment of the Ar group to the main structure of the molecule.

Because of the presence of the tetrahydrofuran ring, compounds of formula (I) possess at least two chiral centres, namely the attachment points of the sulphonamide and the phenyl ring on the tetrahydrofuran ring. The tetrahydrofuran compounds may exist in four stereoisomers—a pair of diastereomers (cis and trans), each comprising a pair of enantiomers with respect to the chiral centres in the tetrahydrofuran.

In one embodiment, compounds of formula (I) have the cis configuration as shown in formula (Ia):

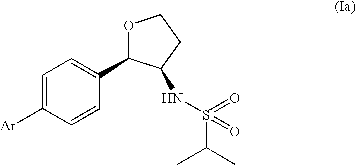

It will be appreciated, in common with most biologically active molecules that the level of biological activity may vary between the enantiomers of a given molecule. It is intended that the scope of the invention includes both enantiomers and all mixtures thereof, including but not limited to racemic mixtures, which demonstrate appropriate biological activity with reference to the procedures described herein.

In one embodiment a compound of the invention in chiral form has at least 80% e.e. In another a compound of the invention in chiral form has at least 90% e.e., for example at least 95% e.e. In another embodiment the isomers correspond to at least 98% e.e, for example at least 99% e.e.

It will be appreciated that the present invention is intended to include compounds having any combination of the groups listed hereinbefore.

It will be understood that, where appropriate, an embodiment described above for one part of the invention may be combined with an embodiment of another part of the invention.

### Examples of compounds of formula (I) include

N-{cis-2-[4-(6-fluoro-3-pyridinyl)phenyl]tetrahydro-3-furanyl}-2-propanesulfonamide
N-{cis-2-[4-(6-methyl-3-pyridinyl)phenyl]tetrahydro-3-furanyl}-2-propanesulfonamide
N-{cis-2-[4-(5-fluoro-2-pyridinyl)phenyl]tetrahydro-3-furanyl}-2-propanesulfonamide
N-{cis-2-[4-(5-fluoro-3-pyridinyl)phenyl]tetrahydro-3-furanyl}-2-propanesulfonamide
N-{cis-2-[4-(5-methyl-3-pyridinyl)phenyl]tetrahydro-3-furanyl}-2-propanesulfonamide
N-[cis-2-(4′-fluoro-4-biphenylyl)tetrahydro-3-furanyl]-2-propanesulfonamide
N-[cis-2-(4′-cyano-4-biphenylyl)tetrahydro-3-furanyl]-2-propanesulfonamide
N-[cis-2-(3′-acetyl-4-biphenylyl)tetrahydro-3-furanyl]-2-propanesulfonamide
N-{cis-2-[4-(2-thienyl)phenyl]tetrahydro-3-furanyl}-2-propanesulfonamide
   and salts and solvates thereof.

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1S)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

Furthermore, some of the crystalline forms of the compounds of structure (I) may exist as polymorphs, which are included in the present invention. Some of the compounds of this invention may be crystallised or recrystallised from solvents such as aqueous and organic solvents. In such cases solvates may be formed. This invention includes within its scope stoichiometric solvates as well as compounds containing variable amounts of solvent, where non-stoichiometric solvates may be produced by processes such as lyophilisation. In one embodiment, the compounds of the present invention are provided in the form of stoichiometric and non-stoichiometric hydrates.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may be administered as prodrugs. Examples of pro-drug forms for certain compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Examples of prodrugs for certain compounds of the invention include: amides, carbamates and phosphamides. The present invention therefore also provides a prodrug of a compound of formula (I) or a salt or solvate thereof.

Hereinafter, compounds of formula (I), their salts and their solvates defined in any aspect of the invention (except Intermediate compounds in chemical processes) are referred to as “compounds of the invention”.

Since the compounds of the invention are intended for use in pharmaceutical compositions it will readily be understood that they may each be provided in substantially pure form, for example at least 85%, or at least 98% pure or at least 99% pure (% are on a weight for weight basis). Impure preparations of the compounds may be used for preparing the more pure forms used in the pharmaceutical compositions.

Compounds of the invention may be prepared in a variety of ways. In the following reaction schemes and hereafter, unless otherwise stated Ar and Y are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of general formula (I) may be prepared from compounds of formula (II) by reaction with isopropyl sulfonyl chloride according to reaction scheme 1. Typical reaction conditions comprise adding isopropyl sulfonyl chloride to a mixture of (II) and 1,8-diazabicyclo[5.4.0]undec-7-ene with cooling.

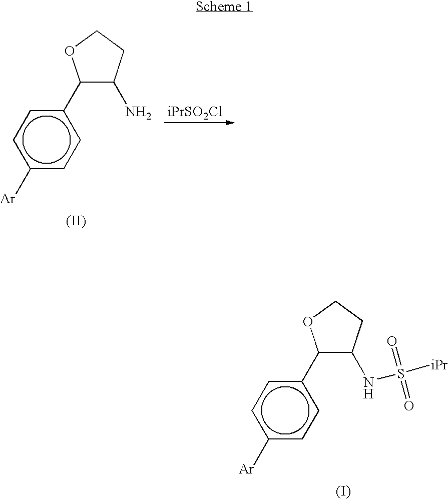

Alternatively, compounds of formula (I) may be prepared by coupling a compound of formula (III) where X is a leaving group such as halogen (for example chlorine, bromine or iodine) with a boronic acid derivative of formula (IV) according to reaction Scheme 2. Typical coupling conditions comprise heating a compound of formula (III), a compound of formula (IV), a base (or potassium fluoride where the leaving group is chlorine), 2-(di-tert-butylphosphino)biphenyl and palladium (II) acetate in dry tetrahydrofuran at about 140 degC. The method of Scheme 2 is particularly suitable for compounds of the invention in which Ar is pyridyl.

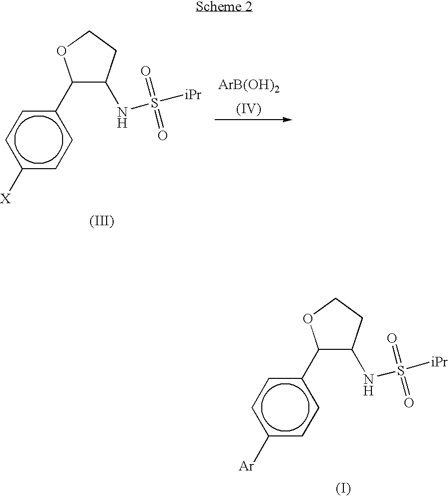

Alternatively, a compound of formula (I) may be prepared by treating a boronate compound of formula (V) with a compound Ar—X (VI) where X is a leaving group such as halogen (for example bromine or iodine) as shown in Scheme 3. For example the boronate may be a 4,4,5,5-tetramethyl-1,3,2-dioxaborolan-2-yl compound as shown in Scheme 3. Typical reaction conditions comprise heating the boronate compound (V) with a compound of formula (VI), a base (or potassium fluoride where the leaving group is chlorine) and a suitable catalyst (for example a palladium catalyst, such as palladium (II) acetate of palladium tetrakis, which may be polymer-supported) in a suitable solvent (for example dry 1,4-dioxane) at for example, about 90° C. Suitable bases include sodium carbonate solution

The boronate compound (V) may be prepared by reaction of a compound of formula (III) with a suitable diboron compound. Typical coupling conditions comprise heating the compound of formula (III) (for example at approximately 90° C.) with the diboron compound (for example bis(pinacolato)diboron) in a suitable solvent (for example dioxane) in the presence of a suitable base (for example potassium acetate) to produce a compound of formula (V).

The method of Scheme 3 is particularly suitable for compounds of the invention in which Ar is phenyl, pyridyl, thienyl or furanyl.

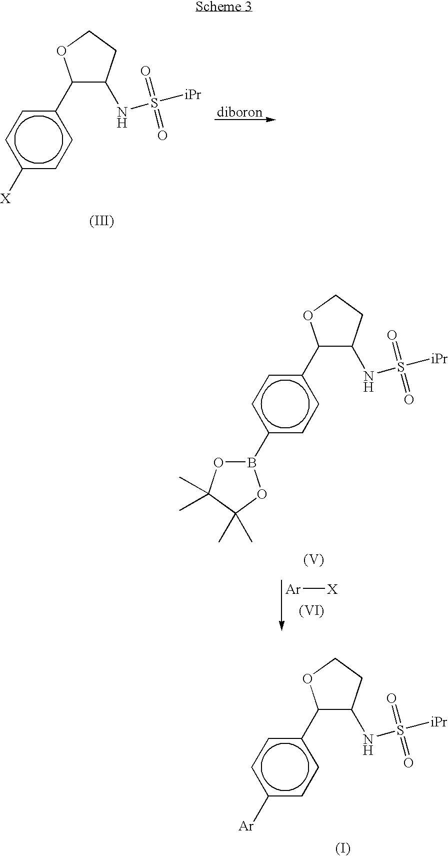

Compounds of general formula (II) (see scheme 1) may be prepared from compounds of formula (VI) according to reaction scheme 4, by reacting compounds of formula (VI) with indium metal at room temperature.

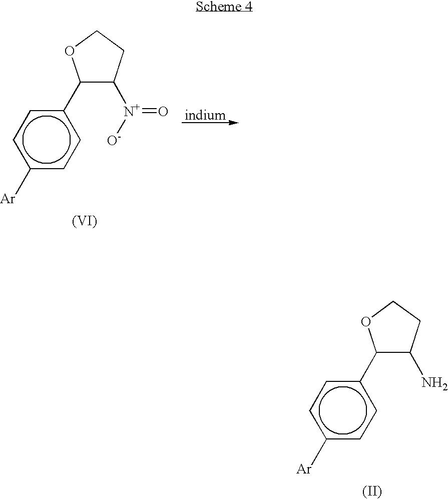

Compounds of formula (III) (see scheme 2 and scheme 3) may be prepared from compounds of formula (VII) according to reaction scheme 5. Typical reaction conditions are sequentially adding a suitable acid (such as trifluoromethanesulfonic acid) and an N-halosuccinimide (VIII) (such as N-iodosuccinimide) to a cooled solution of the compound of formula (VII) in a suitable solvent and then warming the mixture gradually to room temperature.

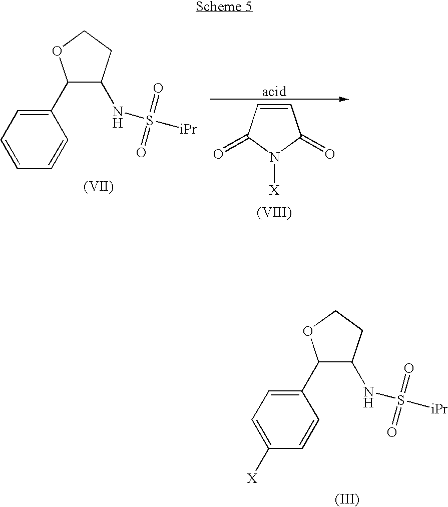

Compounds of formula (VII) may be prepared from compounds of formula (IX) according to reaction scheme 6. Typical reaction conditions are adding isopropyl sulfonyl chloride to an ice-cooled mixture of (IX) and a base (such as diisopropylamine) in a suitable solvent (such as dichloromethane) and then warming the mixture gradually to room temperature.

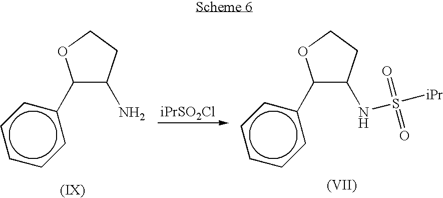

The compound of formula (IX) may be prepared in a sequence of steps from the compound of formula (X), according to scheme 7.

Typical reaction conditions for preparing the compound of formula (IX) comprise the addition of a suitable base (such as sodium hydroxide solution) to a suspension of the compound of formula (XIV) in a solvent (such as dichloromethane).

The compound of formula (XIV) may be prepared by cooling a solution of the compound of formula (XIII) in a suitable solvent (such as dichloromethane) to around −60° C., then adding a saturated solution of ammonia in methanol cooled to −60° C., and allowing the mixture to warm up to room temperature and collecting the resultant crystalline product by filtration. A mixture of cis and trans isomers of (XIV) is expected to be obtained in this step. It is generally possible to isolate one or both of the isomers, for example by crystallisation or by chromatography. It has been found that crystallisation from the reaction mixture of dichloromethane and methanol results in isolation of the cis isomer.

The compound of formula (XIII) may be prepared by adding a suitable base to a cooled solution of the compound of formula (XII) in a suitable solvent (such as dichloromethane), and subsequently adding trifluoromethane sulfonic anhydride. Suitable bases include pyridine with a catalytic amount of dimethylamino pyridine.

The compound of formula (XII) may be prepared by the methods described in US 2005159607 and Organic Preparations and Procedures International (1992), 24(1), 13-20.

Alternatively, the compound of formula (XII) may be prepared by heating a mixture of the compound of formula (XI) and magnesium iodide in a suitable solvent (such as tetrahydrofuran).

The compound of formula (XI) may be prepared by cooling a solution of meta-chloroperbenzoic acid in a suitable solvent (such as dichloromethane), then adding dropwise a solution of the compound of formula (X) in a suitable solvent (such as dichloromethane) and then warming the mixture gradually to room temperature.

The starting material, the compound of formula (X), may be prepared by reduction of trans-styrylacetic acid with a suitable reducing agent (such as lithium aluminium hydride). Typical reaction conditions are adding a solution of trans-styrylacetic acid in a suitable solvent (such as tetrahydrofuran) dropwise to a cooled solution of lithium aluminium hydride in a suitable solvent (such as tetrahydrofuran) and then warming the mixture gradually to room temperature.

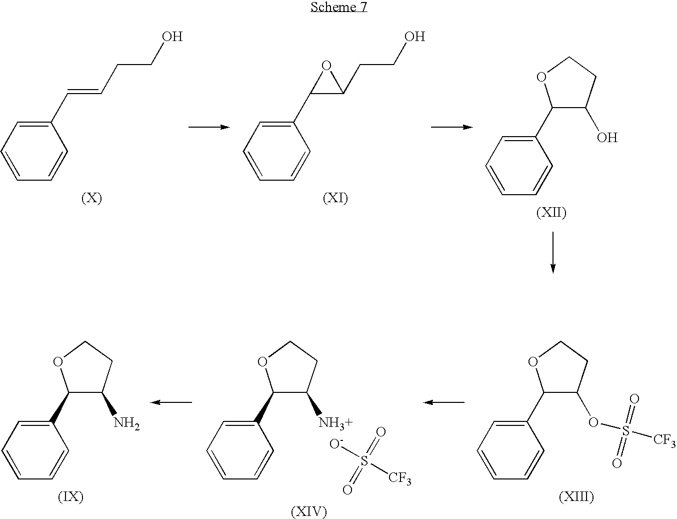

Thus, the present invention also provides a process for preparing a compound of formula (I), the process comprising:

a) reacting a compound of formula (II):

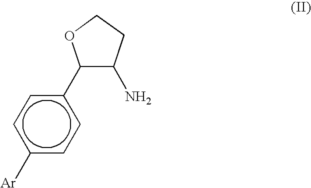

wherein Ar is as defined for formula (I), with isopropylsulfonyl chloride;
or
b) coupling a compound of formula (III) where X is a leaving group such as halogen (for example chlorine, bromine or iodine) with a boronic acid derivative of formula (IV)

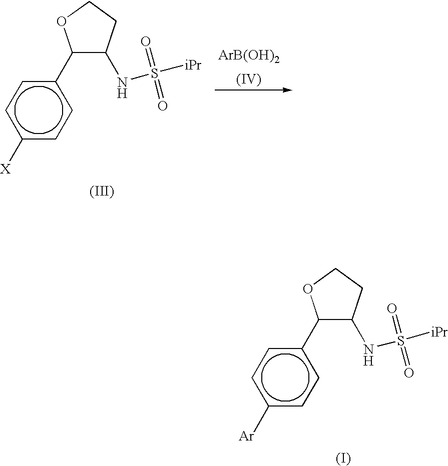

or
c) coupling a boronate compound of formula (V) with a compound Ar—X (VI) where X is a leaving group such as halogen (for example bromine or iodine)

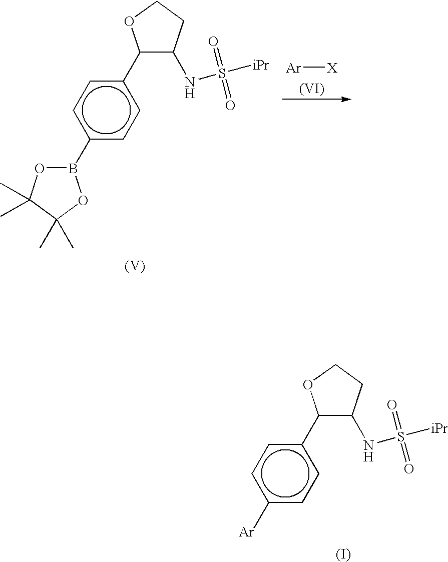

and thereafter optionally:
removing any protecting group(s); and/or
forming a salt or solvate; and/or
converting one compound of formula (I) to a different compound of formula (I).

Further details for the preparation of compounds of formula (I) are found in the Examples section hereinafter.

The compounds of the invention may be prepared singly or as compound libraries comprising at least 2, for example 5 to 1,000 compounds, for example 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

The compounds of the present invention potentiate the glutamate receptor, and are thus considered to be useful for treating diseases and conditions which are mediated by the potentiation of the glutamate receptor.

It will be appreciated that the invention includes the following further aspects. The embodiments described in respect of the first aspect apply equally to each of these further aspects:
i) a pharmaceutical composition comprising a compound of the invention and at least one carrier, diluent or excipient;
ii) the use of a compound of the invention in the manufacture of a medicament for treating a disease or condition mediated by a reduction or imbalance in glutamate receptor function in a mammal;
iii) a compound of the invention for use in treating a disease or condition mediated by a reduction or imbalance in glutamate receptor function in a mammal;
iv) a compound of the invention for use as a medicament;
v) a method of treatment of a disease or condition mediated by a reduction or imbalance in glutamate receptor function in a mammal comprising administering an effective amount of a compound of the invention;
vi) a combination product of a compound of the invention with an antipsychotic;
vii) a pharmaceutical composition comprising a combination product as defined in vi) above and at least one carrier, diluent or excipient;
viii) the use of a combination product as defined in vi) above in the manufacture of a medicament for treating a disease or condition mediated by a reduction or imbalance in glutamate receptor function in a mammal;
ix) a combination product as defined in vi) above for use in treating a disease or condition mediated by a reduction or imbalance in glutamate receptor function in a mammal;
x) a combination product as defined in vi) above for use as a medicament;
xi) a method of treatment of a disease or condition mediated by a reduction or imbalance in glutamate receptor function in a mammal comprising administering an effective amount of a combination product as defined in vi) above.

In the case of aspects ii), iii), v), viii), ix) and xi), relevant diseases or conditions are: psychosis and psychotic disorders (including schizophrenia, schizo-affective disorder, schizophreniform diseases, brief reactive psychosis, child onset schizophrenia, “schizophrenia-spectrum” disorders such as schizoid or schizotypal personality disorders, acute psychosis, alcohol psychosis, drug-induced psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); cognitive impairment (e.g. the treatment of impairment of cognitive functions including attention, orientation, memory (i.e. memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states) trauma, aging, stroke, neurodegeneration, drug-induced states, neurotoxic agents), mild cognitive impairment, age related cognitive impairment, autism related cognitive impairment, Down's syndrome, cognitive deficit related to psychosis, post-electroconvulsive treatment related cognitive disorders; anxiety disorders (including generalised anxiety disorder, social anxiety disorder, agitation, tension, social or emotional withdrawal in psychotic patients, panic disorder, and obsessive compulsive disorder); neurodegenerative diseases (such as Alzheimer's disease, amyotrophic lateral sclerosis, motor neurone disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); post-traumatic stress syndrome; attention deficit disorder; attention deficit hyperactivity disorder; drug-induced (phencyclidine, ketamine and other dissociative anaesthetics, amphetamine and other psychostimulants and cocaine) disorders; Huntingdon's chorea; tardive dyskinesia; dystonia; myoclonus; spasticity; obesity; stroke; sexual dysfunction; sleep disorders, and some forms of epilepsy.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Compounds of the invention may also be of use in the treatment of the following disorders:—

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-induced Disorders such as Substance Intoxication, Substance Withdrawal, Substance-Induced Delirium, Substance-Induced Persisting Dementia, Substance-Induced Persisting Amnestic Disorder, Substance-induced Psychotic Disorder, Substance-Induced Mood Disorder, Substance-Induced Anxiety Disorder, Substance-induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-Induced Mood Disorder, Cocaine-Induced Anxiety Disorder, Cocaine-Induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-induced Psychotic Disorder, Inhalant-induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:

Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).

Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

All of the various forms and sub-forms of the disorders mentioned herein are contemplated as part of the present invention.

Within the context of the present invention, the term “cognitive impairment” includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the invention may be administered in conventional dosage forms prepared by combining a compound of the invention with standard pharmaceutical carriers, diluents or excipients according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, for example water. The compound, depending on the vehicle and concentration used, can be either suspended or dissolved in the vehicle. In preparing solutions the compound can be dissolved in water for injection and filter sterilised before filling into a suitable vial or ampoule and sealing.

Agents such as a local anaesthetic, preservative and buffering agents may also be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. A surfactant or wetting agent may also be included in the composition to facilitate uniform distribution of the compound.

The compositions of the present invention may contain from 0.1% by weight, for example from 10-60% by weight, of the active material, depending on the method of administration. Where the compositions comprise dosage units, each unit may, for example contain from 0.1 to 20 mg of the active ingredient. For example, such a unit may contain from 1 to 10 mg. The dosage as employed for adult human treatment may, for example, range from 2 to 50 mg per day, for instance 5 to 20 mg per day depending on the route and frequency of administration (though in some instances, a dosage of 50 mg to 100 mg per day may be appropriate). Based on a 75 kg individual, such a dosage corresponds to 0.027 to 0.667 mg/kg per day. Suitably the dosage is from 0.05 to 0.3 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e. the number of doses of a compound of the invention given per day for a defined number of days, can be ascertained by those skilled in the art using conventional course of treatment determination tests.

The compounds of the invention may be used in combination with one or more of the following agents to treat psychotic disorders: i) antipsychotics (such as olanzapine, risperidone, clozapine, ziprazidone, talnetant); ii) drugs for extrapyramidal side effects, for example anticholinergics (such as benztropine, biperiden, procyclidine, trihexyphenidyl), antihistamines (such as diphenhydramine), dopaminergics (such as amantadine); iii) antidepressants; iv) anxiolytics; v) cognitive enhancers for example cholinesterase inhibitors (such as tacrine, donepezil, rivastigmine, galantamine).

The compounds of the invention may be used in combination with antidepressants to treat depression and mood disorders.

The compounds of the invention may be used in combination with one or more of the following agents to treat bipolar disease: i) mood stabilisers; ii) antipsychotics; iii) antidepressants.

The compounds of the invention may be used in combination with one or more of the following agents to treat anxiety disorders: i) anxiolytics; ii) antidepressants.

The compounds of the invention may be used in combination with one or more of the following agents to improve nicotine withdrawal and reduce nicotine craving: i) nicotine replacement therapy, for example a sublingual formulation of nicotine beta-cyclodextrin and nicotine patches; ii) drugs for treating nicotine addition, for example bupropion.

The compounds of the invention may be used in combination with one or more of the following agents to improve alcohol withdrawal and reduce alcohol craving: i) NMDA receptor antagonists for example acamprosate; ii) GABA receptor agonists for example tetrabamate; iii) Opioid receptor antagonists for example naltrexone.

The compounds of the invention may be used in combination with one or more of the following agents to improve opiate withdrawal and reduce opiate craving: i) opioid mu receptor agonist/opioid kappa receptor antagonist for example buprenorphine; ii) opioid receptor antagonists for example naltrexone; iii) vasodilatory antihypertensives for example Iofexidine.

The compounds of the invention may be used in combination with one or more of the following agents to treat sleeping disorders: i) benzodiazepines for example temazepam, lormetazepam, estazolam, triazolam; ii) non-benzodiazepine hypnotics for example zolpidem, zopiclone, zaleplon, indiplon; iii) barbiturates for example aprobarbital, butabarbital, pentobarbital, secobarbita, phenobarbital; iv) antidepressants; v) other sedative-hypnotics for example chloral hydrate, chlormethiazole.

The compounds of the invention may be used in combination with one or more of the following agents to treat anorexia: i) appetite stimulants for example cyproheptidine; ii) antidepressants; iii) antipsychotics; iv) zinc; v) premenstrual agents for example pyridoxine and progesterones.

The compounds of the invention may be used in combination with one or more of the following agents to treat bulimia: i) antidepressants; ii) opioid receptor antagonists; iii) antiemetics for example ondansetron; iv) testosterone receptor antagonists for example flutamide; v) mood stabilisers; vi) zinc; vii) premenstrual agents.

The compounds of the invention may be used in combination with one or more of the following agents to treat autism: i) antipsychotics; ii) antidepressants; iii) anxiolytics; iv) stimulants for example methylphenidate, amphetamine formulations, pemoline.

The compounds of the invention may be used in combination with one or more of the following agents to treat Attention Deficit Hyperactivity Disorder: i) stimulants for example methylphenidate, amphetamine formulations, pemoline; ii) non-stimulants for example norepinephrine reuptake inhibitors (such as atomoxetine), alpha 2 adrenoceptor agonists (such as clonidine), antidepressants, modafinil, cholinesterase inhibitors (such as galantamine and donezepil).

The compounds of the invention may be used in combination with one or more of the following agents to treat personality disorders: i) antipsychotics; ii) antidepressants; iii) mood stabilisers; iv) anxiolytics.

The compounds of the invention may be used in combination with one or more of the following agents to treat male sexual dysfunction: i) phosphodiesterase V inhibitors, for example vardenafil, sildenafil; ii) dopamine agonists/dopamine transport inhibitors for example apomorphine, buproprion; iii) alpha adrenoceptor antagonists for example phentolamine; iv) prostaglandin agonists for example alprostadil; v) testosterone agonists such as testosterone; vi) serotonin transport inhibitors for example serotonin reuptake inhibitors; v) noradrenaline transport inhibitors for example reboxetine; vii) 5-HT1A agonists, for example flibanserine.

The compounds of the invention may be used in combination with one or more of the following agents to treat female sexual dysfunction: i) the same agents specified for male sexual dysfunction, ii) an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone, amisulpride, ziprazidone and talnetant).

Antidepressant drugs include serotonin reuptake inhibitors (such as citalopram, escitalopram, fluoxetine, paroxetine and sertraline); dual serotonin/noradrenaline reuptake inhibitors (such as venlafaxine, duloxetine and milnacipran); Noradrenaline reuptake inhibitors (such as reboxetine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It is to be understood that “treatment” as used herein includes prophylaxis as well as alleviation of established symptoms.

### EXAMPLES

The invention is illustrated by the Examples described below.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent.

NMR spectra were obtained at 298K, at the frequency stated using either a Bruker™ DPX400 or an Oxford Instruments™ 250 MHz machine and run as a dilute solution of CDCl₃ unless otherwise stated. All NMR spectra were reference to tetramethylsilane (TMS δ_{H} 0, δ_{C} 0). All coupling constants are reported in hertz (Hz), and multiplicities are labelled s (singlet), bs, (broad singlet), d (doublet), t (triplet), q (quartet), dd (doublet of doublets), dt (doublet of triplets) and m (multiplet).

Total ion current traces were obtained for electrospray positive and negative ionisation (ES+/ES−) and atmospheric pressure chemical positive and negative ionisation (AP+/AP−).

### ABBREVIATIONS

THF Tetrahydrofuran

In the procedures that follow, reference to an Intermediate or Example by number is typically provided. This is provided merely for assistance to the skilled chemist to identify the starting material used. The starting material may not necessarily have been prepared from the batch referred to.

### INTERMEDIATES

### Intermediate 1: (3E)-4-phenyl-3-buten-1-ol

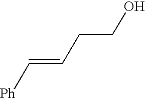

Lithium aluminium hydride (28.0 g, 0.75 mol, 1.05 eq.) was placed in a 5 L flange lid flask fitted with magnetic stirrer, dropping funnel and argon inlet. THF (500 ml) was then added and mixture cooled with an ice-bath. A solution of trans-styrylacetic acid (120.0 g, 0.74 mol, 1.0 eq.) was dissolved in THF (1.5 L) and added dropwise over 1.5 h. After the addition was complete, the reaction mixture was allowed to warm up to R.T. The reaction was worked-up after 45 mins. 1M NaOH (1.5 L) was added slowly which resulted in a gel like mixture. Extra water (300 ml) and ethyl acetate (500 ml) was added to aid separation. The aqueous is emulsion like, but organic layer can be separated. The aqueous layer is then extracted further with ethyl acetate (3×1 L). The combined organics is dried over Na₂SO₄, concentrated under reduced pressure to give the title compound (84 g, 77%) as an orange oil.

### Intermediate 2: 2-(3-phenyl-2-oxiranyl)ethanol

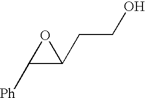

meta-Chloroperbenzoic acid (title ˜70%, 169.0 g, 0.69 mol, 1.2 eq.) was placed in a 5 L flange flask fitted with dropping funnel. Dichloromethane (1.5 L) was added and the white suspension was cooled with and ice-bath. A solution of (3E)-4-phenyl-3-buten-1-ol (Intermediate 1) (85.0 g, 0.57 mol, 1.0 eq.) in dicholomethane (700 ml) was added dropwise. After addition was complete, the ice-bath was removed and allowed to warm up to R.T. TLC (Rf 0.3, 7:3 Pet.ether 40-60/ethyl acetate) showed that reaction was complete after 1 h. The reaction mixture was carefully washed with 10% Na₂SO₃ until starch iodide test proved negative. The organic layer was then separated and washed with sodium bicarbonate (3×1 L), dried over MgSO₄ and concentrated under reduced pressure to give the title compound (84 g, 89%) as an orange oil.

### Intermediate 3: 2-phenyltetrahydro-3-furanol

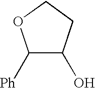

2-(3-phenyl-2-oxiranyl)ethanol (Intermediate 2) (72.0 g, 0.43 mol, 1.0 eq.) was dissolved in THF (900 ml) in a 2 L 3-neck round bottomed flask, with an argon inlet and condenser. Magnesium iodide (2.40 g, 0.0086 mol, 0.02 eq) was added in one portion and mixture heated for 54 h (in oil bath at 75° C.). The reaction mixture was allowed to cool to room temperature, and then organic washed with water (2×300 ml). The aqueous layer was extracted with ethyl acetate (2×500 ml). The combined organics were washed with brine (300 ml), dried over MgSO₄ and concentrated under reduced pressure to give the title compound (72.0 g, 100%, 1:1.8, cis:trans mixture) as an orange oil.

Intermediate 3 is known from US 2005159607 and Organic Preparations and Procedures International (1992), 24(1), 13-20.

### Intermediate 4: 2-phenyltetrahydro-3-furanyl Trifluoromethanesulfonate

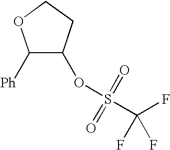

2-phenyltetrahydro-3-furanol (Intermediate 3) (10.0 g, 0.06 mol, 1.0 eq.) was dissolved in dichloromethane (70 ml) in a 500 ml flask with a dropping funnel and argon inlet. This was cooled to −10 to −20° C. using a cardice/acetone bath. Pyridine (14.5 g, 0.18 mol, 3.0 eq.) was then added, followed by a catalytic amount of dimethylamino pyridine (0.4 g, 0.003 mol, 0.05 eq.). Trifluoromethane sulfonic anhydride (25.8 g, 0.09 mol, 1.5 eq.) was then added dropwise over 15 mins, maintaining the temperature at −10 and −20° C. The reaction was stirred at this temperature for 15 mins and water (30 ml) was added. The layers were separated. The aqueous layer was further extracted with dichloromethane (3×60 ml). The combined organics were then washed with 5% aqueous HCl (70 ml), 5% aqueous NaHCO₃ (70 ml), water (70 ml) and brine (70 ml). The organic layer was then dried over Na₂SO₄ to give the title compound in dichloromethane solution which was used immediately in the next step.

### Intermediate 5: cis-2-phenyltetrahydro-3-furanaminium Trifluoromethanesulfonate

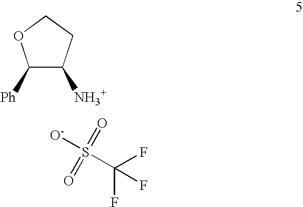

A dichloromethane solution of 2-phenyltetrahydro-3-furanyl trifluoromethanesulfonate (intermediate 4) (250 ml) prepared as described above was placed in a 1 L flask. This was then cooled to −60° C. Meanwhile, methanol (100 ml) was placed in a three-necked round bottomed flask fitted with cold-finger, thermoprobe and dip tube connected to an ammonia cylinder. The methanol was then cooled to <−60° C. using cardice/acetone bath and ammonia was bubbled through the solution until saturated with ammonia. This solution was then added to the 1 L flask. Additional ammonia was added into the solution via a dip tube at −60° C. The solution was then allowed to warm up to room temperature. and left to stir overnight.

4 runs of the above preparation were combined and concentrated under reduced pressure to yield a dark red slurry. This was then triturated with chloroform (75 ml) and resulting solid isolated by filtration. This was washed with a minimal amount of chloroform to yield the title compound (28.5 g) as a tan solid. A second crop was obtained by concentrating the filtrate and treating with chloroform (7.5 g, Total 36 g, 47% from intermediate 4).

¹H-NMR (400 MHz, DMSO d⁶) δ: 7.35 (m, 5H), 7.10 (m, 3H), 4.84 (d, 1H), 4.10 (m, 1H), 3.95 (m, 1H), 3.81 (m, 1H), 2.38 (m, 1H), 1.99 (m, 1H)

### Intermediate 6: cis-2-phenyltetrahydro-3-furanamine

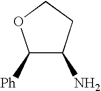

cis-2-phenyltetrahydro-3-furanaminium trifluoromethanesulfonate (Intermediate 5) (10 g, 0.32 mol) was suspended in dichloromethane (100 ml). 0.5M NaOH (100 ml) was then added and mixtured stirred until solid dissolved. The layers were then separated and the organic layer was extracted with dichloromethane (4×100 ml). 20% aq. NaOH (a few drops) was added to ensure the pH remained at 14. The combined organics were dried over Na₂SO₄ and concentrated to near dryness under reduced pressure. The amount of the title compound was calculated by ¹H nmr (˜3.6 g, 69%). The cis arrangement of the Ph and NH₂ groups was confirmed by an NMR NOE experiment.

### Intermediate 7: cis-Propane-2-sulfonic Acid (2-phenyltetrahydrofuran-3-yl)amide

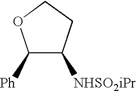

cis-2-phenyltetrahydro-3-furanamine (Intermediate 6) (4.6 g, 0.028 mol, 1.0 eq.) was dissolved in dichloromethane in a flask, fitted with condenser and argon inlet. This was cooled with an ice-bath. Triethylamine (5.7 g, 0.056 mol, 2.0 eq.) and dimethylaminopyridine (0.35 g, 0.0028 mol, 0.1 eq.) were then added, followed by isopropyl sulfonyl chloride (8.0 g, 0.056 mol, 2.0 eq.). The ice-bath was removed and the mixture was allowed to warm to room temperature. A precipitate was formed after ˜5 mins. Dichloromethane (25 ml) was added and the solid re-dissolved. This was then heated to 34° C. and left to stir overnight. The reaction mixture was allowed to cool to room temperature. The solvent was then reduced by 2/3 volume, where a solid precipitated. This was isolated by filtration and washed with the minimal amount of CH₂Cl₂. The filtrate was then washed with water (2×15 ml). The combined aqueous portions were extracted with dichloromethane (4×100 ml). The combined organics were washed with brine (15 ml), dried over MgSO₄ and concentrated under reduced pressure. A suction column was performed using 9:1 Pet. ether 40-60/EtOAc, Product Rf=0.3 in 1:1 Pet. ether 40-60/ethyl acetate.

A flash column was performed on the combined columned products from all the previous runs, using 90:10 Pet. ether 40-60/ethyl acetate as eluent, to give the title compound (2.2 g, 9%) as a yellow solid.

¹H-NMR (400 MHz, DMSO d⁶) δ: 7.27 (m, 4H), 7.18 (m, 1H), 6.81 (d, 1H), 4.73 (d, 1H), 4.08 (m, 2H), 3.74 (m, 1H), 2.61 (m, 1H), 2.28 (m, 1H), 1.88 (m, 1H), 0.92 (d, 3H), 0.77 (d, 3H). The cis arrangement of the Ph and NHSO₂iPr groups was confirmed by an NMR NOE experiment.

### Intermediate 8: cis-N-[2-(4-iodophenyl)tetrahydro-3-furanyl]-2-propanesulfonamide

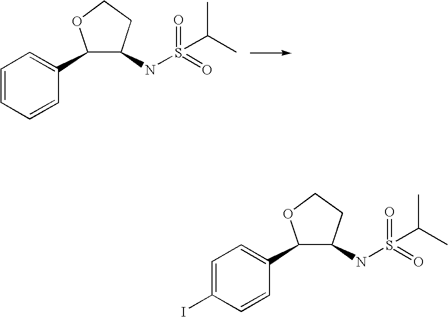

cis-Propane-2-sulfonic acid (2-phenyltetrahydrofuran-3-yl)amide (Intermediate 7) (2.1 g, 7.8 mmol) was dissolved in dry acetonitrile (40 ml), then N-iodosuccinimide (1.6 g, 7.1 mmol, 0.9 eq) was added and the solution cooled to 0° C. After addition of trifluoromethanesulfonic acid (2.0 ml, 21.8 mmol, 2.8 eq), the ice-bath was removed and the mixture stirred at room temperature overnight. Further portions of N-iodosuccinimide (0.8 g, 3.55 mmol, 0.45 eq) and trifluoromethanesulfonic acid (1.0 ml, 10.9 mmol, 1.4 eq) were added and the mixture was stirred for 22 hrs at room temperature. The reaction mixture was diluted in ethyl acetate/saturated aqueous NaHCO₃. The organic layer was separated and the aqueous phase was further extracted with ethyl acetate. The combined organic layers were dried and concentrated.

The crude product was purified by flash chromatography, eluting with 30% ethyl acetate in cyclohexane, to give the title compound as a yellowish solid (868 mg, 28% yield).

¹HNMR (DMSO) δ: 7.7 (2H, d), 7.15 (2H, d), 6.95 (1H, d), 4.75 (1H, s), 4.15 (2H, m), 3.8 (1H, m), 2.8 (1H, m), 2.3 (1H, m), 1.9 (1H, m), 1.05 (3H, d), 0.9 (3H, m).

The mixture of products was analysed by chiral HPLC:

### Chiral Chromatographic Conditions

| | |
|---|---|
| Column: | Chiralpak AS-H, 25 × 4.6 mm |
| Mobile phase: | n-Hexane 30%, ethanol 70% |
| Flow rate: | 0.8 ml/min |
| CD | 235 nm |
| DAD | 220 nm |

Rt (enantiomer 1) 9.750 min, area % 52.47. Rt (enantiomer 2) 41.213 min, area % 45.13.

### Intermediate 9: cis-N-{2-[4-(4,4,5,5-tetramethyl-1,3,2-dioxaborolan-2-yl)phenyl]tetrahydro-3-furanyl}-2-propanesulfonamide

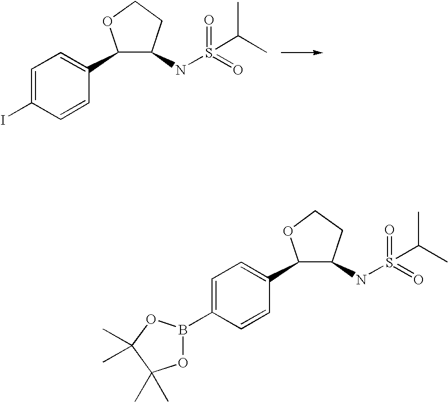

cis-N-[2-(4-iodophenyl)tetrahydro-3-furanyl]-2-propanesulfonamide (Intermediate 8) (810 mg, 2.05 mmol) was dissolved in dry dimethyl sulfoxide (10 ml). Potassium acetate (604 mg, 6.15 mmol, 3 eq), 1,1′-Bis(diphenylphosphino)ferrocene-palladium(II)dichloride (82 mg, 0.10 mmol, 0.05 eq) and bis(pinacolato)diboron (625 mg, 2.46 mmol, 1.2 eq) were added to the solution. The reaction mixture was heated at 80° C. for 4 hrs with stirring and then left overnight at room temperature.

Further portions of potassium acetate (604 mg, 6.15 mmol, 3 eq), 1,1′-Bis(diphenylphosphino)ferrocene-palladium(II)dichloride (82 mg, 0.10 mmol, 0.05 eq) and bis(pinacolato)diboron (625 mg, 2.46 mmol, 1.2 eq) were added. The mixture was stirred at 90° C. for 3 hrs and left overnight at room temperature.

The reaction was filtered and the solid residue washed with ethyl acetate. The resulting solution was concentrated and re-dissolved in water/ethyl acetate. The organic layer was separated and the aqueous phase was further extracted with ethyl acetate (2×). The combined organic layers were dried and concentrated.

The crude product was purified by flash chromatography, eluting with 30% ethyl acetate in cyclohexane, to give the title compound as a yellow oil (664 mg, 82% yield).

¹HNMR (DMSO) δ: 7.7 (2H, d), 7.3 (2H, d), 6.9 (1H, d), 4.8 (1H, s), 4.15 (2H, m), 3.8 (1H, m), 2.8 (1H, m), 2.3 (1H, m), 1.95 (1H, m), 1.3 (6H, s), 1.15 (6H, s), 1.05 (3H, d), 0.9 (3H, m).

### General Procedure to Examples 1 to 9

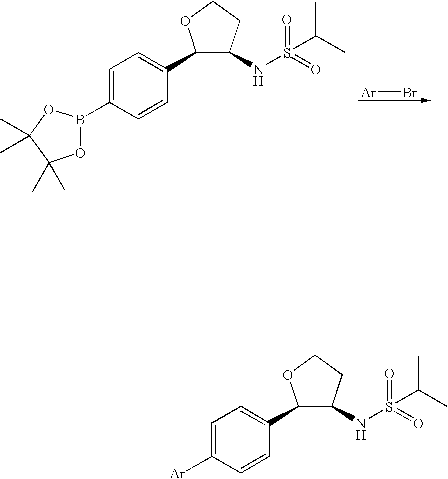

The compounds of examples 1 to 9 were prepared in an array.

In 8 ml vials the bromoaryl reagent (0.36 mmol, 2 eq) was placed and a solution of cis-N-{2-[4-(4,4,5,5-tetramethyl-1,3,2-dioxaborolan-2-yl)phenyl]tetrahydro-3-furanyl}-2-propanesulfonamide (Intermediate 9) (73 mg, 0.18 mmol, 1.0 eq) in dry 1,4-dioxane (1.0 ml), polymer supported Tetrakis catalyst (49 mg, 0.0054 mmol, 0.03 eq, loading 0.11 mmol/g), and aqueous 2M Na₂CO₃ (225 uL, 0.45 mmol, 2.5 eq) were added. The reaction mixture was shaken at 90° C. for 4.5 hrs. The resin was filtered off and washed with dichloromethane, methanol, and water. The resulting solution was evaporated to dryness with a blow-down apparatus and the residue was partitioned between dichloromethane (3 ml) and 3N aqueous HCl. The organic layer was separated on a hydrophobic frit cartridge, the aqueous phase was basified with aqueous 2M NaOH and extracted with dichloromethane (2×3 ml). The combined organic extracts were concentrated using a blow-down apparatus to obtain a crude compound which was purified by mass directed reverse phase HPLC to give the title compound in each case for Examples 1 to 9. The HPLC conditions were as given in “Preparative Chromatography” above.

Examples 1 to 9 as synthesised by this route are summarised in Table 1:

TABLE I

| | | | Mol. Formula | LC-MS Rt | |
|---|---|---|---|---|---|
| Ex | Structure | Name | Calc MW | (min)/(M+) | Yield |
| 1 | 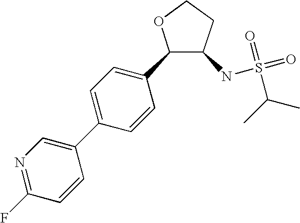 | N-{cis-2-[4-(6-fluoro-3- pyridinyl)phenyl]tetrahydro- 3-furanyl}-2- propanesulfonamide | C₁₈H₂₁FN₂O₃S 364.4 | 2.67 365 | 26.3 mg 40.1% |
| 2 | 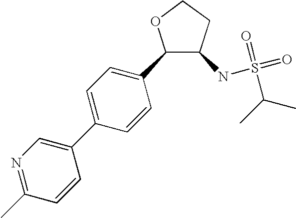 | N-{cis-2-[4-(6-methyl-3- pyridinyl)phenyl]tetrahydro- 3-furanyl}-2- propanesulfonamide | C₁₉H₂₄N₂O₃S 360.5 | 2.26 361 | 17.3 mg 26.7% |
| 3 | 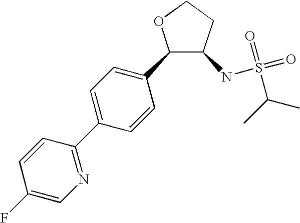 | N-{cis-2-[4-(5-fluoro-2- pyridinyl)phenyl]tetrahydro- 3-furanyl}-2- propanesulfonamide | C₁₈H₂₁FN₂O₃S 364.4 | 2.69 365 | 10 mg 15.2% |
| 4 | 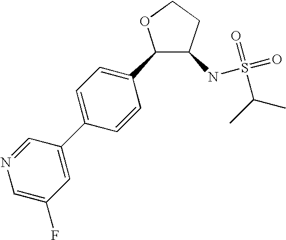 | N-{cis-2-[4-(5-fluoro-3- pyridinyl)phenyl]tetrahydro- 3-furanyl}-2- propanesulfonamide | C₁₈H₂₁FN₂O₃S 364.4 | 2.42 365 | 20.1 mg 30.6% |
| 5 | 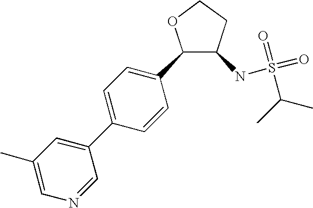 | N-{cis-2-[4-(5-methyl-3- pyridinyl)phenyl]tetrahydro- 3-furanyl}-2- propanesulfonamide | C₁₉H₂₄N₂O₃S 360.5 | 2.61 361 | 5.9 mg 9.1% |
| 6 | 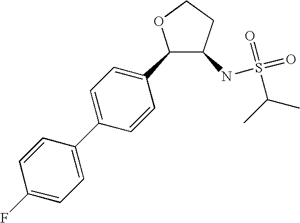 | N-[cis-2-(4′-fluoro-4- biphenylyl)tetrahydro-3- furanyl]-2- propanesulfonamide | C₁₉H₂₂FNO₃S 363.5 | 3.54 | 36.2 mg 55.3% |
| 7 | 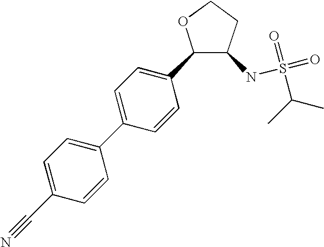 | N-[cis-2-(4′-cyano-4- biphenylyl)tetrahydro-3- furanyl]-2- propanesulfonamide | C₂₀H₂₂N₂O₃S 370.5 | 3.17 371 | 14.2 mg 21.3% |
| 8 | 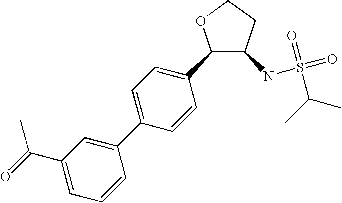 | N-[cis-2-(3′-acetyl-4- biphenylyl)tetrahydro-3- furanyl]-2- propanesulfonamide | C₂₁H₂₅NO₄S 387.5 | 3.04 388 | 23.2 mg 33.3% |
| 9 | 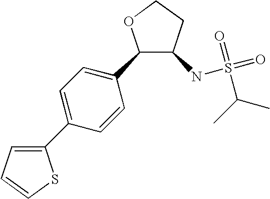 | N-{cis-2-[4-(2- thienyl)phenyl]tetrahydro- 3-furanyl}-2- propanesulfonamide | C₁₇H₂₁NO₃S₂ 351.5 | 3.36 352 | 29.2 mg 46.2% |

Examples 6 and 9 were analysed by ¹H-NMR, giving the following spectral data:

### Example 6

¹HNMR (DMSO) δ: 7.70 (2H, m), 7.60 (2H, d), 7.41 (2H, d), 7.26 (2H, t), 6.96 (1H, d), 4.84 (1H, d), 4.18 (2H, m), 3.80 (1H, m), 2.74 (1H, m), 2.35 (1H, m), 1.91 (1H, m), 1.00 (3H, d), 0.82 (3H, d).

### Example 9

¹HNMR (DMSO) δ: 7.60 (2H, d), 7.51 (2H, m), 7.35 (2H, d), 7.12 (1H, dd), 6.94 (1H, d), 4.79 (1H, d), 4.16 (2H, m), 3.78 (1H, m), 2.76 (1H, m), 2.32 (1H, m), 1.95 (1H, m), 1.03 (3H, d), 0.88 (3H, d). The cis configuration at the tetrahydrofuran ring was confirmed by an NOE NMR experiment.

### Analytical HPLC Conditions (Used for Analysis Quoted in Table 1)

| | |
|---|---|
| Column 1: | GEMINI C18 5 μm, 50 × 4.6 mm |
| Mobile phase | A: NH4HCO3 sol. 10 mM, pH 10; B: CH₃CN |
| Gradient: | 35% (B) for 0.5 min, from 35% (B) to |
| | 95% (B) in 4.5 min, 95% (B) for 1.5 min |
| Flow rate: | 2 ml/min |
| UV wavelength range: | 210-350 nm |
| Mass range: | 100-900 amu |
| Ionization: | ES+ |

### Preparative Chromatographic Conditions

| | |
|---|---|
| Column: | GEMINI C18 5 μm, 100 × 21 mm |
| Mobile phase: | A: NH4HCO3 sol. 10 mM, pH 10; B: CH3CN |
| Gradient 1*: | 35% (B) for 1 min, from 35% (B) to |
| | 60% (B) in 9 min, 100% (B) for 5 min |
| Gradient 2**: | 50% (B) for 1 min, from 50% (B) to |
| | 85% (B) in 9 min, 100% (B) for 5 min |
| Flow rate: | 17 ml/min |
| UV wavelength range: | 210-350 nm |
| Mass range: | 100-900 amu |
| Ionization: | ES+ |

*used for Examples 1, 2, 3, 4, 5
**used for Examples 6, 7, 8, 9

### Biological Assays

The ability of the compounds of the invention to potentiate glutamate receptor-mediated response may be determined a) by using fluorescent calcium-indicator dyes such as FLUO4 and additionally b) by measuring glutamate-evoked current recorded from human GluR2 flip unedited HEK293 cells.

### a) Calcium Influx Fluorescence Assay

384 well plates are prepared containing confluent monolayer of HEK 293 cells either stably expressing or transiently transfected with human GluR2 flip (unedited) AMPA receptor subunit. These cells form functional homotetrameric AMPA receptors. The tissue culture medium in the wells are discarded and the wells are each washed three times with standard buffer (80 μL) for the stable cell line (145 mM NaCl, 5 mM KCl, 1 mM MgCl₂, 2 mM CaCl₂, 20 mM N-[2-hydroxyethyl]-piperazine-N-[2-ethanesulfonic acid (HEPES), 5.5 mM glucose, pH 7.3) or with a Na-free buffer for the transient transfected cells (145 mM N-methyl-glucamine instead of NaCl). The plates are then incubated for 60 minutes in the dark with 2 μM FLUO4-AM dye (20 μL) (Molecular Probes, Netherlands) at room temperature to allow cell uptake of the FLUO-4AM, which is then converted to FLUO-4 by intracellular esterases which is unable to leave the cell. After incubation each well is washed three times with buffer (80 μL) (30 μL of buffer remained in each well after washing).

Compounds of the invention (or reference compounds such as cyclothiazide) are dissolved in dimethylsulfoxide (DMSO) at a stock concentration of 10 mM. These solutions are further diluted with DMSO using a Biomek FX (Beckman Coulter) in a 384 compound plate. Each dilution (1 μL) is transferred to another compound plate and buffer (50 μL) is added. An agonist stimulus (glutamate) plate is prepared by dissolving sodium glutamate in water to give a concentration of 100 mM. This solution is diluted with buffer to give a final concentration of 500 μM and dispensed into another 384-well plate (50 μL/well) using a Multidrop (Thermolabsystems).

The cell plate is then transferred into a fluorescence imaging plate based reader [such as the FLIPR384 (Molecular Devices)]. A baseline fluorescence reading is taken over a 10 to 240 second period, and then 10 μL from each plate containing a compound of the invention made up in standard buffer solution (in a concentration range from 100 μM to 10 pM) is added (to give a final concentration in the range 30 μM to 3 pM). The fluorescence is read over 5 minute period. 500 μM glutamate solution (10 μL) is added (to give a final concentration of 100 μM). The fluorescence is then read over a 4 minute period. The activities of the compounds of the invention and reference compounds are determined by measuring peak fluorescence after the last addition. The activity is also expressed relative to the fluorescence increase induced by cyclothiazide at their maximum response (i.e. greater than 30 μM).

The assay described above is believed to have an effective limit of detection of a pEC₅₀ in the region of 3.5-4.0 due to the limitations of compound solubility. The pEC₅₀ result is generally considered to be accurate +/−0.3. Accordingly, a compound exhibiting a pEC₅₀ value within this range from such an assay may indeed have a reasonable affinity for the receptor, but equally it may also have a lower affinity, including a considerably lower affinity.

The Example compounds were screened using the assay as described above and Examples 1 to 9 gave a pEC₅₀ equal to or greater than 4.0 and demonstrated an activity at least 50% that of cyclothiazide (at its maximal response).

### b) Whole Cell Voltage-Clamp Electrophysiology Assay

The ability of the compounds of the invention to potentiate AMPA-subtype glutamate receptor-mediated response are determined by measuring AMPA-evoked current recorded from rat cultured hippocampal neurons.

This assay involves the electrophysiological characterisation of AMPA receptor positive modulators using rat cultured hippocampal neurons. The extracellular recording solution contains: 145 mM NaCl, 2.5 mM KCl, 1.2 mM MgCl₂, 1.5 mM CaCl₂, 10 mM N-[2-hydroxyethyl]-piperazine-N-[2-ethanesulfonic acid (HEPES), 10 mM D-glucose, pH 7.3 with NaOH. The intracellular solution contains: 80 mM CsCl, 80 mM CsF, 10 mM N-[2-hydroxyethyl]-piperazine-N-[2-ethanesulfonic acid (HEPES), 10 mM ethylene glycol-bis(g-aminoethylether)-N,N,N′,N,-tetra-acetic acid (EGTA), 14 mM MgATP, 14 mM DiTris Creatine Phosphate, 50 U/ml Creatine Phosphokinase pH 7.3 with CsOH. Recording electrodes are prepared from glass capillary tubes (Clark Electromedical GC120-F10) pulled into two equal lengths using a Zeitz Instruments DMZ Universal Puller, program 09, resulting in electrodes with a resistance of approximately 3-6 MOhms when measured in extracellular solution. Electrodes are back filled with internal recording solution. Positive pressure is applied to the electrode to prevent mixture of internal and external solutions and assist in formation of high resistance seal when the electrode makes contact with the cell membrane. Glass coverslip fragment, bearing rat cultured hippocampal neurons, is placed in the recording chamber positioned on the stage of an inverted microscope. A tube at the edge of the chamber is used to apply extracellular solution to the bath. Rapid solution exchange uses a fast step perfusion system (Biologic RSC160). Two outlet tubes attached together along their length are positioned close to a chosen cell so that the outflow from only one tube can pass directly over the cell surface. A motorized stepper could re-position the tubes such that the outflow from the second outlet tube flows over the cell allowing solution exchange at the cell membrane surface to occur within 10-20 ms. Excess bath solution is removed via a tube positioned at the edge of the chamber connected to a vacuum line.

A prospective cell is positioned in the centre of the microscope field of view. Recording electrode is positioned directly above the cell membrane surface. Using fine manipulator control (Luigs and Neumann, SM-6) the electrode is lowered, while monitoring the change in electrode resistance during delivery of a 5 mV depolarizing pulse, until a high resistance seal (gigaseal) is achieved. Whole cell configuration is achieved by removing by suction a small fragment of cell membrane immediately beneath the recording electrode tip. The cell membrane potential is held at −70 mV (voltage-clamped) via the electrode (Axopatch 200B Integrating patch clamp amplifier, pClamp software, Axon Instruments). Test solutions are applied using the fast application system using the following protocol and changes in inward current are recorded and stored for off-line analysis.

1) Control current−exchange from extracellular solution to extracellular solution+30 μM AMPA (2 s application time, 30 s interval between applications) repeated until measurements are stable.

2) Test current−exchange from extracellular solution+10 nM of compound of invention to extracellular solution+10 nM of compound of invention+30 μM AMPA (2 s application time, 30 s interval between applications) repeated until measurements are stable.

All experiments are performed at ambient temperature (20 to 22° C.).

The activity of a compound of the invention is determined by measuring the area under the curve (during 2 s period of application) for the 30 μM AMPA response in the presence of the compound of the invention and expressing it as % of potentiation of the 30 μM AMPA alone response (30 μM AMPA in the absence of the compound of the invention).

1. A compound of formula (I), or a salt, or solvate thereof: 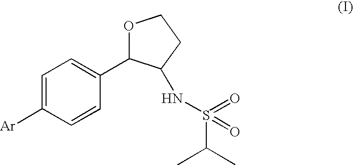
wherein:
Ar is selected from the group consisting of phenyl, pyridyl, furanyl and thienyl, each optionally substituted with one or more groups Y;
each Y group is independently selected from the group consisting of: halo, C₁₋₄alkyl, haloC₁₋₄alkyl, C₁₋₄alkoxy, cyano, C(O)C₁₋₄alkyl, NHSO₂C₁₋₄alkyl, NMeSO₂C₁₋₄alkyl, NHCOC₁₋₄alkyl, NMeCOC₁₋₄alkyl, SOC₁₋₄alkyl, SO₂C₁₋₄alkyl, and CO₂C₁₋₄alkyl, or two Y groups together form a cyclic group —O(CH₂)O—.

2. A compound, or a salt or solvate thereof as claimed in claim 1 which is a compound of formula (Ia), or a salt or solvate thereof: 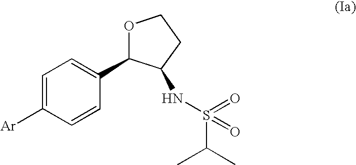
wherein:
Ar is selected from the group consisting of phenyl, pyridyl, furanyl and thienyl, each optionally substituted with one or more groups Y;
each Y group is independently selected from the group consisting of: halo, C₁₋₄alkyl, haloC₁₋₄alkyl, C₁₋₄alkoxy, cyano, C(O)C₁₋₄alkyl, NHSO₂C₁₋₄alkyl, NMeSO₂C₁₋₄alkyl, NHCOC₁₋₄alkyl, NMeCOC₁₋₄alkyl, SOC₁₋₄alkyl, SO₂C₁₋₄alkyl, and CO2C₁₋₄alkyl, or two Y groups together form a cyclic group —O(CH₂)O—.

3. A compound as claimed in claim 1, wherein the compound is selected from:
N-{cis-2-[4-(6-fluoro-3-pyridinyl)phenyl]tetrahydro-3-furanyl}-2-propanesulfonamide
N-{cis-2-[4-(6-methyl-3-pyridinyl)phenyl]tetrahydro-3-furanyl}-2-propanesulfonamide
N-{cis-2-[4-(5-fluoro-2-pyridinyl)phenyl]tetrahydro-3-furanyl}-2-propanesulfonamide
N-{cis-2-[4-(5-fluoro-3-pyridinyl)phenyl]tetrahydro-3-furanyl}-2-propanesulfonamide
N-{cis-2-[4-(5-methyl-3-pyridinyl)phenyl]tetrahydro-3-furanyl}-2-propanesulfonamide
N-[cis-2-(4′-fluoro-4-biphenylyl)tetrahydro-3-furanyl]-2-propanesulfonamide
N-[cis-2-(4′-cyano-4-biphenylyl)tetrahydro-3-furanyl]-2-propanesulfonamide
N-[cis-2-(3′-acetyl-4-biphenylyl)tetrahydro-3-furanyl]-2-propanesulfonamide
N-{cis-2-[4-(2-thienyl)phenyl]tetrahydro-3-furanyl}-2-propanesulfonamide
and salts and solvates thereof.

4. A pharmaceutical composition comprising a compound as claimed in claim 1 and at least one pharmaceutically acceptable carrier or diluent.

5-10. (canceled)

11. A method of treatment or prevention of a disease or condition mediated by a reduction or imbalance in glutamate receptor function in a mammal comprising administering an effective amount of a compound as claimed in claim 1.

12. A method as claimed in claim 11 wherein the disease is schizophrenia.

13. A method as claimed in claim 11 wherein the disease is impairment of cognition.

14. A combination product comprising a compound as claimed in claim 1, with an antipsychotic.

15. A process for preparing a compound as claimed in claim 1, the process comprising:
a) reacting a compound of formula (II): 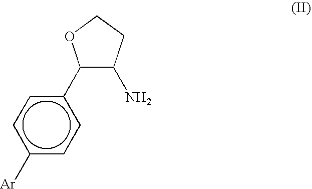
wherein Ar is as defined for formula (I), with isopropylsulfonyl chloride;
or
b) coupling a compound of formula (III) where X is a leaving group such as halogen (for example chlorine, bromine or iodine) with a boronic acid derivative of formula (IV) 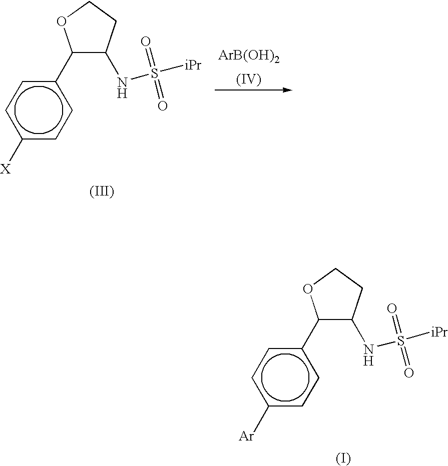
or
c) coupling a boronate compound of formula (V) with a compound Ar—X (VI) where X is a leaving group such as halogen (for example bromine or iodine) 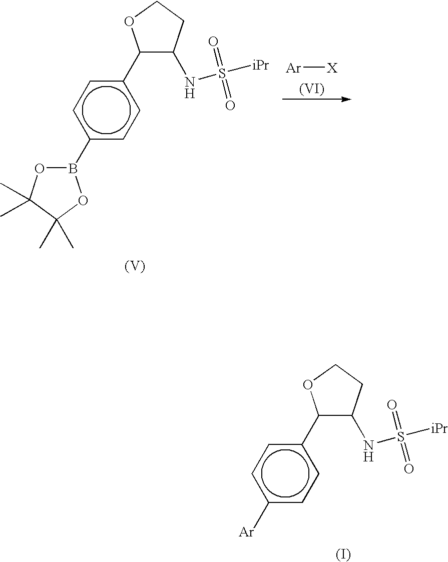
and thereafter optionally:
removing any protecting group(s); and/or
forming a salt or solvate; and/or
converting one compound of formula (I) to a different compound of formula (I).
